(19) United States
(12) Patent Application Publication
(10) Pub. No.: US 2004/0260171 A1
(43) Pub. Date: Dec. 23, 2004

(54) Combined tomography and radiographic projection system

(76) Inventor: Rainer Graumann, Hochstadt (DE)

Correspondence Address: SCHIFF HARDIN LLP, Patent Department, 6600 Sears Tower, 233 South Wacker Drive, Chicago, IL, 60606, US

(21) Appl. No.: 10/824,235

(22) Filed: Apr. 14, 2004

(30) Foreign Application Priority Data
Apr. 14, 2003 (DE) 103 17 132.0

(51) Int. Cl. G21K 1/12; A61B 6/00; G01T 1/166; A61B 5/05

(52) U.S. Cl. 600/411; 250/363.04; 600/427; 600/436

(57) ABSTRACT

A combined apparatus has an apparatus to acquire tomographic image datasets and an apparatus to acquire radiographic projection images. A first examination region in which radiographic projection images are acquired includes at least a part of a second examination region in which the tomographic image datasets are acquired. A number of radiographic projection images can be acquired from which a three-dimensional reconstruction of the first examination region is possible.

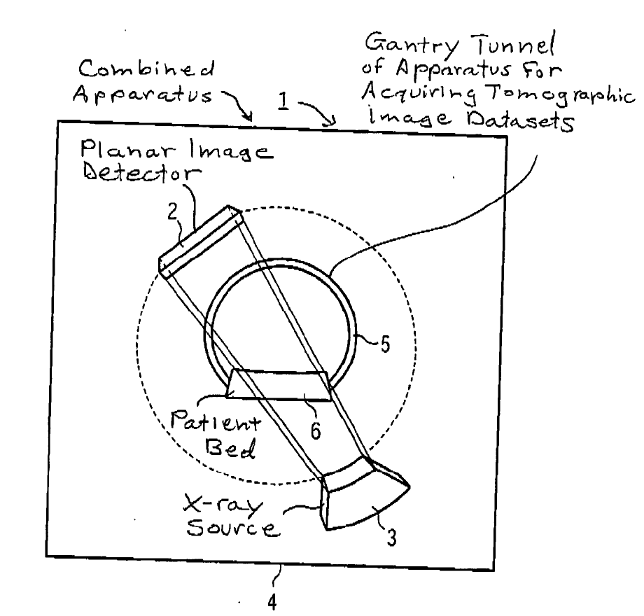

### BACKGROUND OF THE INVENTION

1. Field of the Invention

The present invention concerns an apparatus for combined recording of three-dimensional, functional and structural anatomic image data.

2. Description of the Prior Art

Functional anatomical image data refers to representations of organ functions such as, for example, metabolic events, biochemical reactions or the like. Appropriate examinations are primarily effected in cardiology, neurology and oncology. Three-dimensional image datasets are typically acquired with tomographic acquisition methods. These tomography methods based on very different physical procedures, are generally referred to below as functional tomography.

In nuclear-medical diagnostics, the patient is injected with specific unstable nuclides that accumulate in specific organs. By detection of the corresponding decay products of the preparation emitted from the body, an image is obtained of the spatial distribution (dispersal) forming the basis of biochemical events. The metabolic preparations include preferred gamma quanta or positron emitters.

The image generation ensues with different methods depending on the type of radioactive decay. For example, given the use of individual photon emitters, meaning nuclides that decay with emission of individual gamma quanta, the slice imaging method SPECT (Single Photon Emission Computed Tomography) is used. For this, the measurement head of a gamma camera are in general moved on an orbit around the patient. The acquisition of projections of the activity distribution on the measurement plane of the gamma camera ensues from discrete angles. In general, a camera with a large-area (surface area) measurement head is used, such that projections of volumes can be acquired.

PET (positron emission tomography) is used for imaging with positron emitters, meaning nuclides that decay under emission of positrons. The positrons are indirectly accounted for by the gamma quanta emitted in the opposite direction upon annihilation. A number of detectors are arranged around the patient in a circle for spatial imaging. The activity distribution in the examination region can thus be reconstructed from the measured values along the connecting line between two detectors (LOR, line of response).

In the recent past, a further special field of radiology, interventional radiology, has emerged. For this purpose, functional magnetic resonance (fMR) images are obtained that show activity that occurs in the tissue instead of the morphology of the tissue. This application of fMR normally ensues in the field of neurology or neurosurgery.

The specified methods yield a spatial distribution of specific organ functions, known as functional data. They provide scant additional structural data, meaning image information about the organs in which the examined activity or the examined process occurs. For improved, reliable diagnoses, and for significant improvement of therapy planning, functional data must be combined with structural data.

Presently, structural information is for the most part acquired with different devices, separate from the functional information. In practice, essentially two approaches to link these separately generated image datasets have become accepted. In the simplest case, it is incumbent upon the medical personnel viewing the images to achieve, by mental effort, the nature of the association in between the separately generated image datasets. The reliability of this mental correlation of the two image datasets is substantially determined by the experience of the diagnostician and his or her current concentration capability.

In order to achieve a certain independence from these human factors, in the second case the separately registered image datasets are fused into a common representation, so the individual images can be both superimposed and displayed next to one another. A disadvantage of this technique is that the patient must be transferred between the two different apparatuses, resulting in practice in a non-negligible position change of the organs from the first exposure to the second exposure, which must be calculated.

A third approach uses a combination apparatus, for example a PET-CT combination. In which the image datasets can be generated from an identically positioned patient. As described in U.S. Pat. No. 6,490,476, this can be achieved by a suitable, adjacent arrangement of a PET apparatus with a computed tomography apparatus in connection with a specially fashioned patient bed usable in both apparatuses. The position of the patient in the two examination apparatuses can be precisely monitored, such that the temporally separately acquired image datasets can be spatially associated.

U.S. Pat. No. 6,449,331 adopts another approach. A detector is used that is able to detect both gamma quanta and x-ray radiation. A functional exposure and a structural exposure of a specific examination region on the patient thus can be generated without having to move the patient between the exposures.

However, it is to be expected that a common detector solution represents a measurement-technical compromise via which each imaging modality is separately limited. In general, the use of a CT apparatus in combination with a PET apparatus results in an operating system that is markedly expensive. Furthermore, the common use of important components of such a device combination makes a separation and a separate use of the involved imaging modalities impossible.

The combination of computed tomography (CT) with positron emission tomography (PET), or with SPEC tomography (SPECT), represents a very cost-intensive imaging method, and the measurement time, in particular for PET and SPECT, is—in contrast to CT—relatively long (in the minute range). A relatively high radiation exposure of the patient is associated with this longer time, and an examination of the patient in an uncomfortable position (such as, for example, with arms extended over the head) is not possible given such long measurement times. Thus, in specific examinations of functional organ dysfunctions, no correlated acquisition of functional and structural anatomical image data can be effected, since a position change of the patient from one measurement to the other always leads to a shifting of the organs.

The combination of functional tomography with conventional computed tomography would lead to much shorter measurement times (in the second range), but heretofore a combination of this type has been considered prohibitively cost-intensive and additionally very complex with regard to the success of the combination capability.

### SUMMARY OF THE INVENTION

It is an object of the present invention to provide an apparatus for spatially correlated generation of three-dimensional functional and structural anatomical image datasets that avoids the disadvantages of the prior art cited above.

This object is achieved in accordance with the invention comprising a combined an apparatus having a device to acquire radiographic projection images in a first examination region and a device to acquire tomographic image datasets in a second examination region. The first examination region in which the radiographic projection images are acquired includes at least part of the second examination region in which the tomographic image dataset are acquired. Furthermore, a number of selected radiographic projection images can be obtained so that, from these projection images, a three-dimensional reconstruction of the first examination region is possible that can be correlated with the second examination region based on the fixed spatial relation between the two regions.

Since, with a device to acquire radiographic projection images, the projection images necessary for a three-dimensional reconstruction can be acquired in a very short time (meaning in less than a minute), it is possible to register anatomical structures without placing unreasonable requirements on the patient or his or her position. The combination in one apparatus of a device to acquire tomographic image datasets with a device to generate radiographic projection images allows exposures of an examination region to be made with different techniques but with a fixed spatial relation.

For the examination of biochemical processes, the device to acquire tomographic image datasets is appropriately a particle emission-tomographic system. Dependent on the diagnostic task, a SPECT (Single Photon Emission Computed Tomography) apparatus or a PET (Positron Emission Tomography) apparatus can be used as a particle emission-tomographic acquisition system.

For interventional procedures, a tomography system for functional magnetic resonance can be used as the apparatus for acquisition of tomographic image datasets.

The apparatus for acquisition of radiographic projection images can be a C-arm x-ray apparatus, such that no development costs are necessary for the design of the movement of the x-ray projection apparatus. A C-arm represents a substantially simpler imaging apparatus (in comparison with a gantry-type CT apparatus) and can be more easily combined with other imaging apparatuses.

The apparatus for the acquisition of radiographic projection images preferably is disposed outside of the housing of the apparatus for the acquisition of tomographic image datasets, but with a fixed spatial relation to housing. A combination apparatus thus can be created from two existing apparatus types with simple means.

In a preferred embodiment, the apparatus for the acquisition of radiographic projection images is integrated into a common housing together with the apparatus for the acquisition of tomographic image datasets, such that a compact combination apparatus results.

### DESCRIPTION OF THE DRAWINGS

The single FIGURE shows an inventive combination apparatus with a system, superior to a PET system, to acquire radiographic projection images from different angles.

### DESCRIPTION OF THE PREFERRED EMBODIMENTS

In the most recent past, the development of C-arm x-ray systems has advanced so far that not only is a three-dimensional reconstruction of high-contrast subjects (such as, for example, bones of vessels filled with contrast agent) possible, but also three-dimensional representations of low-contrast subjects (such as, for example, organs) can be determined from the projection exposures. This is substantially due to improvements in planar image detectors and the evaluation methods used. C-arm x-ray systems thus can provide structure information of organs.

In order to obtain an image dataset for a three-dimensional representation of a measurement subject, a series of projection images of the measurement subject must be acquired from different spatial directions. The acquisition time is short, with typical times of 15 to 20 seconds. This is primarily because the technique of obtaining individual slices obtained with linearly arranged detectors is no longer used, but instead a planar image detector with which a complete projection image can be acquired in one pass of the radiation source and detector is used. The image quality is, in fact, clearly less than that which is achievable with a computer tomography system, but it is more than sufficient in order to provide structural information about organs for which anatomical functional measurements are to be effected.

In an apparatus to acquire radiographic projection images, a planar image detector 2 is disposed opposite the emission opening of an x-ray source 3, such that the x-rays are incident on the x-ray-sensitive surface of the detector 2. In the exposures, the patient or the examination subject is located between the x-ray source 3 and the planar image detector 2. The subject is irradiated, and x-ray absorption distributions are acquired as projection images. In order to acquire sufficient data for a three-dimensional reconstruction of the examination subject, the arrangement composed of the planar image detector 2 and the x-ray source 3 rotates around the examination subject. Up to 200 projection exposures are acquired in equal or variable angle increments. Using a projection matrix describing the image geometry, an image dataset with a three-dimensional representation of the irradiated subject is generated from these projection exposures in a known manner.

The apparatus to acquire radiographic projection images therefore is suited to acquire information about the anatomical structures of a patient in the shortest time. There is no need to undertake a corresponding measurement simultaneously with the measurement of functional anatomic processes. Typically, corresponding measurements with a SPECT, PET or MR system take some minutes. In contrast to this, the radiographic exposure to determine the anatomical structures lasts approximately one minute, and thus represents (when effected separately) no significant extension of the total measurement time. A reliable measurement of the anatomical structures therefore can be acquired with a radiographic examination just before or after the corresponding functional examination.

The FIGURE shows an inventive combination apparatus 1. It is composed of a PET system 4 having a gantry tunnel 5, in front of which is mounted a unit composed of a planar image detector 2 and an x-ray source 3 opposite thereto. The unit composed of the planar image detector 2 and the x-ray source 3 rotates around a common rotation axis that substantially coincides with the axis of symmetry of the gantry tunnel 5. Instead of being mounted at the entrance of the gantry tunnel 5 of a PET system 4, the unit composed of the planar image detector 2 and the x-ray source 3 can be mounted at the entrance of the gantry tunnel of a SPECT or MR system.

For the examination, a patient is positioned on the patient bed 6. This bed 6 can be moved, monitored with regard to position, parallel to the tunnel axis of the gantry tunnel 5. The examination region of the patient is brought into the beam path of the radiographic apparatus composed of the planar image detector 2 and the x-ray source 3 either before or after the functional tomography. For the measurement, the radiographic apparatus is rotated around its rotation axis through a defined angle range, during which radiographic projection exposures are acquired at defined angle increments. The measurement volume from which the data are acquired is a cube approximately 30 cm long on each side, so that in practice the entire examination region is acquired with a single radiographic exposure series. A spatial association of both image datasets is achieved by knowledge (by monitoring) of the shift path of the patient bed between the radiographic exposure and the functional exposure. This is sufficient since the patient does not have to be transferred and the shifting time is short enough in relation to the total measurement time so as to exclude patient movements.

As an alternative to the arrangement shown in the FIGURE, a C-arm x-ray apparatus can be fixedly mounted in front of the gantry of a functional tomography apparatus, meaning a PET, SPECT or MR system. A fixed spatial relation between the radiographic and the functional measurement region is given by the fixed mounting. The equipment of the functional tomography apparatus with its own mechanism for isocentric rotation of the unit composed of the planar image detector 2 and the x-ray source 3 can be advantageously foregone with this solution, so a significant cost savings can be achieved, particularly given low production numbers. If the connection between the C-arm x-ray apparatus and the functional tomograph is detachable, both apparatuses can be used independently of one another as needed. A number of diagnostic methods thus can be implemented with flexible apparatus use.

It is also possible, however, to integrate the radiographic apparatus into a common housing with the functional tomographic apparatus. This solution is particularly preferable for particle emission-tomographic systems and when the combination apparatus is predominantly used to examine organ functions with simultaneous acquisition of the anatomical structures. The integration enables both detector systems, meaning the detector system for the acquisition of the decay products and the planar image detector, to be arranged adjacent to one another. The patient no longer has to be moved a short distance between the two measurements. Furthermore, it is also possible to tilt the radiographic apparatus with regard to its rotation axis, such that the connection line between the planar image detector 2 and the x-ray source 3 permeates the measurement region of the emission-tomographic detector. A shifting of the patient between the two measurements thus is unnecessary, and both measurements can possibly ensue within the same time span.

The above-described combination, in one apparatus, of an apparatus to acquire tomographic image datasets and an apparatus to acquire radiographic projection images allows multi-modal imaging to associate functional anatomical information with structural anatomical information for diagnosis, therapy planning and therapy support. Due to the substantially short measurement times (with regard to CT combination apparatuses), the radiation exposure as well as the emotional stress on the patient is substantially reduced. In addition, the above-described system can be substantially more cost-effectively produced than a comparable CT combination apparatus.

Although modifications and changes may be suggested by those skilled in the art, it is the intention of the inventor to embody within the patent warranted hereon all changes and modifications as reasonably and properly come within the scope of his contribution to the art.

I claim as my invention:

1. A combined apparatus comprising:
an apparatus for acquiring radiographic projection images in a first examination region;
an apparatus for acquiring tomographic image datasets in a second examination region;
said first examination region of said apparatus for acquiring radiographic projection images including at least a part of said second examination region of said apparatus for acquiring tomographic image datasets, giving said first and second examination regions a fixed spatial relation to each other; and
said apparatus for acquiring radiographic projection images being operable to acquire a plurality of radiographic projection images in said first examination region and for generating, from said plurality of radiographic projection images, a three-dimensional reconstruction of said first examination region correlatable with said second examination region based on said fixed spatial relation.

2. A combined apparatus as claimed in claim 1 wherein said apparatus for acquiring tomographic image datasets is a particle emission tomographic acquisition system.

3. A combined apparatus as claimed in claim 2 wherein said particle emission-tomographic acquisition system is a single photon emission computed tomography system.

4. A combined apparatus as claimed in claim 2 wherein said particle emission-tomographic acquisition system is a positron emission tomography system.

5. A combined apparatus as claimed in claim 1 wherein said apparatus for acquiring tomographic image datasets is a functional magnetic resonance apparatus.

6. A combined apparatus as claimed in claim 1 wherein said apparatus for acquiring radiographic projection images is a 3D C-arm x-ray apparatus.

7. A combined apparatus as claimed in claim 1 wherein said apparatus for acquiring tomographic image datasets has a housing, and wherein said apparatus for acquiring radiographic projection images is disposed outside of said housing and in a fixed spatial relation to said housing.

8. A combined apparatus as claimed in claim 7 comprising a common housing containing both said apparatus for acquiring radiographic projection images and said apparatus for acquiring radiographic projection images.
